SEQUENCE LISTING (1 sequence)

SEQ ID NO: 1 - protein, 1132 aa, Homo sapiens [sequence omitted]

(19) United States
(12) Patent Application Publication
(10) Pub. No.: US 2005/0136539 A1
(43) Pub. Date: Jun. 23, 2005

(54) Reversible immortalization of human renal proximal tubular epithelial cells

(76) Inventors: Claudia M. Kowolik, Redondo Beach, CA (US); Jiing-Kuan Yee, Arcadia, CA (US)

Correspondence Address: PERKINS COIE LLP, POST OFFICE BOX 1208, SEATTLE, WA, 98111-1208, US

(21) Appl. No.: 10/962,380

(22) Filed: Oct. 8, 2004

Related U.S. Application Data
(60) Provisional application No. 60/510,386, filed on Oct. 8, 2003.

(51) Int. Cl. C12N 15/867; C12N 5/08

(52) U.S. Cl. 435/456; 435/369

(57) ABSTRACT

The present provides reversibly immortalized RPTECs and methods for making and utilizing these cells. Specifically, the present invention provides a method of reversibly immortalizing RPTECs by introducing a first vector containing a human telomerase catalytic subunit (hTERT) gene flanked by loxP sites and a second vector containing an SV40 T antigen (Tag) gene flanked by loxP sites. Immortalization can be reversed by introduction of a third vector containing a Cre recombinase or Cre variant gene. The reversibly immortalized RPTECs generated by this method may be used for a variety of applications, including screening of test agents for the ability to modulate renal toxicity or incorporation into devices designed to mimic the activity of the renal proximal tubules.

### RELATED APPLICATIONS

The present utility application claims priority to U.S. Provisional Application No. 60/510,386 (Kowolik et al.), filed Oct. 8, 2003, the disclosure of which is incorporated by reference herein in its entirety.

### GOVERNMENT INTEREST

This invention was made with government support in part by grants from the National Institutes of Health. The government may have certain rights in this invention.

### FIELD OF THE INVENTION

The present invention relates to the fields of cell immortalization and renal cell function, specifically renal proximal tubular epithelial cell function.

### BACKGROUND OF THE INVENTION

Primary human cells are useful both in research and in applications such as drug toxicity screening and cell-based therapies (Salmon 2000; Noguchi 2002). However, primary cells are restricted in their use by limited proliferation potential and by variability in donor genetic background, gender, and age (Racusen 1997; Kobayashi 2000a). In addition, culturing primary cells is expensive.

One reason for the limited proliferation potential of primary cells is progressive telomere shortening occurring at each cell division, which ultimately leads to cell senescence (Harley 1990; Wright 1992a). This limitation can be overcome by the expression of polyoma or SV40 T antigen (Tag), which allows the cells to progress through an additional number of cell divisions before a second, irreversible crisis occurs (Wright 1989; Shay 1993). A few cells can overcome the second crisis and eventually acquire the ability to grow indefinitely in culture (Rhim 1985; Yoakum 1985; Hurlin 1989; Wright 1992b; Burger 1998). This process, whereby a primary cell harvested in vivo is induced to grow indefinitely in vitro, is referred to as cell immortalization. Primary rodent cells can be readily immortalized, but human cells are generally more refractory to this manipulation (Land 1983; Ruley 1983; Stevenson 1986; Prowse 1995; Bodnar 1998). The difference in telomere length between rodents and humans may play an important role in their respective ability to be immortalized (Macieira-Coelho 1988; Sachsinger 2001).

Recent studies demonstrate that human cells can be transformed in a stepwise process, requiring the combined expression of a series of growth-promoting and anti-senescence genes (Hahn 1999; DiRenzo 2002; Lundberg 2002). Thus, through proper genetic manipulation, it may be possible to induce controlled proliferation of human cells in culture (Westerman 1996; Kobayashi 2000a; Salmon 2000). Establishing immortalized cell lines that are phenotypically stable and retain all or most primary cell functions could potentially overcome the limitations of primary cells. Several studies have demonstrated the possibility of establishing immortalized human cell lines in culture. Expression of the human telomerase catalytic subunit (hTERT) can prevent growth arrest and immortalize human fibroblasts and epithelial cells (Herbert 2002; Kim 2002). While these immortalized cells actively proliferated in culture, their growth requirements, cell-cycle checkpoints, and karyotypic stability were similar to normal parental cells. However, only a limited number of cells could be immortalized by expression of hTERT alone (Bodnar 1998; Jiang 1999; Morales 1999; Condon 2002; Kawano 2003). Most cell types required the expression of a second gene such as the gene encoding SV40 Tag (Kiyono 1991; O'Hare 2001).

Continuous proliferation of immortalized cells in culture frequently down-regulates the expression of tissue-specific genes when compared with their primary cell counterparts (Racusen 1997; Kim 2000). The presence of genes for cell immortalization precludes the application of these cells in cell-based therapies due to safety concerns. It has been reported that the ectopic expression of hTERT in combination with two oncogenes (SV40 large-T oncoprotein and an oncogene allele of H-ras) results in direct tumorigenic conversion of normal human epithelial and fibroblast cells.

There is a need in the art for novel methods of inducing controlled proliferation of human cells in culture through proper genetic manipulation (Westerman 1996; Berghella 1999; Kobayashi 2000b). Ideally, these methods will allow for the repression or removal of the production or function of proteins responsible for triggering the cell immortalization so that immortalized cells may resume the functions of their primary counterparts without undergoing tumorigenic conversion.

Renal proximal tubule epithelial cells (RPTECs) are cells that line the surface of the renal proximal tubules. These cells are involved in the transport of various substances between blood and urine in the proximal tubules. RPTECs are an important target for a variety of nephrotoxic medicines and other agents that induce renal toxicity (van de Water 2001). Damage to RPTECs in response to severe sepsis or septic shock is a primary factor in development of acute tubular necrosis (Racusen 1995). Based on their role in kidney function and toxicity, RPTECs are attractive candidates for reversible immortalization. Immortalized RPTECs could be used in lieu of primary RPTECs to screen various agents for the ability to induce or reverse renal toxicity, or they could be incorporated into devices designed to mimic the physiological role of the renal proximal tubules.

### SUMMARY OF THE INVENTION

The limited proliferation potential of primary human cells currently precludes their use in many applications. Methods of immortalizing primary human cells by expression of hTERT or hTERT in conjunction with one or more other genes have been developed to overcome this limitation, but these methods come with drawbacks of their own. The present invention discloses novel methods for reversibly immortalizing RPTECs.

In one aspect, the present invention provides a method for reversible immortalization of RPTECs. In this method, an RPTEC is immortalized by introduction of a first and second vector. The first vector contains a polynucleotide encoding hTERT, while the second vector contains a polynucleotide encoding SV40 T antigen. The polynucleotide encoding hTERT and the polynucleotide encoding SV40 Tag are both flanked by loxP sites. In one embodiment, the first and second vectors are contained in a single vector or are the same vector. In another embodiment, the first and second vectors are introduced into the RPTEC either simultaneously or sequentially. In another embodiment, the first and second vectors are retroviral vectors, preferably lentiviral vectors. In a preferred embodiment, a third vector containing a polynucleotide encoding Cre or a Cre variant is introduced into the reversibly immortalized RPTEC. The polynucleotide encoding Cre is flanked by loxP sites. In this embodiment, Cre or the Cre variant is transiently expressed in the cell. In certain embodiments, the third vector is a retroviral vector, preferably a lentiviral vector.

In another aspect, the present invention provides a reversibly immortalized RPTEC into which a first and second vector have been introduced. The first vector contains a polynucleotide encoding hTERT, while the second vector contains a polynucleotide encoding SV40 T antigen. The polynucleotide sequence encoding hTERT and the polynucleotide sequence encoding SV40 Tag are both flanked by loxP sites. In one embodiment, the first and second vectors are contained in a single vector or are the same vector. In another embodiment, the first and second vectors are retroviral vectors, preferably lentiviral vectors. In a preferred embodiment, a third vector containing a polynucleotide encoding Cre or a Cre variant has been introduced into the reversibly immortalized RPTEC. In this embodiment, the polynucleotide encoding Cre or a Cre variant is flanked by loxP sites, and Cre or a Cre variant is transiently expressed in the cell. In one embodiment, the third vector is a retroviral vector, preferably a lentiviral vector. In another preferred embodiment, the reversibly immortalized RPTEC is used to screen a test agent to determine whether the test agent modulates renal toxicity. In yet another preferred embodiment, the reversibly immortalized RPTEC is incorporated into an artificial renal tubule device.

In another aspect, the present invention provides a method of screening a test agent to determine whether the test agent modulates renal toxicity. In this method, the test agent is contacted to one or more reversibly immortalized RPTECs into which a first and second vector have been introduced. The first vector contains a polynucleotide encoding hTERT, while the second vector contains a polynucleotide encoding SV40 T antigen. The polynucleotide sequence encoding hTERT and the polynucleotide sequence encoding SV40 Tag are both flanked by loxP sites. After contacting with the test agent, the immortalized cell(s) is monitored for changes in function, activity, or viability. A change in any of these characteristics indicates that the test agent induces renal toxicity.

Other aspects or embodiments of the present invention are described in the specification, drawings, examples, and claims herein.

### BRIEF DESCRIPTION OF THE DRAWINGS

FIG. 1: Vector structures. (A) HIV7/CNPO-hTERT plasmid. (B) HIV7/CNPO-Tag plasmid. (C) RV-cre plasmid. CMV/LTR: fusion between the CMV IE gene enhancer and the promoter of the HIV 5′LTR, in which the HIV sequence upstream of the TATA box was replaced by the CMV enhancer. cPPT: 190-bp central polypurine tract sequence from HIV-1. [symbol] : loxP site. IRES: internal ribosome entry site. WPRE: 600-bp post-transcriptional regulatory element from Woodchuck hepatitis virus. ΔU3: 400-bp deletion in the 3′ LTR that completely removed the enhancer and promoter sequences in the U3 region of the HIV 3′ LTR. Arrows indicate the direction of transcription.

FIG. 2: Cell morphology. (A) Primary RPTECs at passage seven. (B) Primary RPTECs at passage seven stained for senescence-specific β-Gal expression. (C) TH1 cells at passage 30. (D) Immunofluorescent staining of cells from (C) for cytokeratin. (E) Proliferation of TH1 and TH2 cells.

FIG. 3: Telomerase and T antigen expression in TH clones. (A) Pool 1 and pool 2 are pooled neo^{R} clones derived from RPTECs transduced with both the Tag and hTERT vectors. Tag3 is a clone derived from RPTECs transduced with the Tag vector alone. TH1, TH3, TH7, and TTH10 are individual clones derived from RPTECs transduced with both Tag and hTERT vectors. RPTEC represents the activity in primary cells. +: with heat treatment; −: no heat treatment. (B) Immunofluorescent staining of TH1 cells for Tag expression.

FIG. 4: Ultrastructure of TH1 cells. (A) Multiple dome formation in TH1 cells (100×). (B) Dome formation at higher magnification (400×). (C) Ultrastructure of TH1 cells (16525×). mv: microvilli; mi: mitochondria. (D) Ultrastructure of TH cells at a higher magnification (38750×), tj: tight junction. (E) Transmission electron micrograph of TH1 cells at passage six. (F) Transmission electron micrograph of TH1 cells at passage 18.

FIG. 5: Growth rate of TH1 cells. (A) Immunofluorescent staining for Tag expression ten days after RV-cre treatment. (B) Growth curves of mock-, RV-GFP-, and RV-cre-treated TH1 cells. (C) Cell morphology three weeks after RV-cre treatment. (D) Staining for senescence-specific β-gal expression three weeks after RV-cre treatment.

FIG. 6: Biochemical and functional analysis of TH cells. (A) AP activity. (B) GGT activity. (C) AMG uptake. (D) Ammonia production from primary RPTECs at various pH. (E) Ammonia production at pH 6.9 with or without RV-cre treatment as indicated.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it us understood that such equivalent embodiments are to be included herein.

### Abbreviations

The following abbreviations are used herein: AP, alkaline phosphatase; bp, base pair; CMV, cytomegalovirus; DMEM, Dulbecco's Modified Eagle's Medium; FCS, fetal calf serum; GFP, green fluorescent protein; GGT, γ-glutamyl transpeptidase; IE, immediate early; IRES, internal ribosome entry site; kDa, kilodalton; MLV, murine leukemia virus; MOI, multiplicity of infection; REGM, Renal Epithelial Cell Growth Medium; RPTEC, renal proximal tubule epithelial cell; Tag, SV40 T antigen.

### Definitions

The term “human telomerase catalytic subunit” or “hTERT” as used herein refers to a polypeptide sequence possessing telomerase catalytic activity. Preferably, this polypeptide sequence has the amino acid sequence set forth in SEQ ID NO. 1 (Meyerson 1997).

The term “vector” as used herein refers to a vehicle containing a polynucleotide sequence of interest, wherein the polynucleotide sequence of interest encodes a polypeptide. The polynucleotide sequence of interest may be operably linked to a promoter, allowing the sequence to be expressed in a host cell into which the vector is introduced. Examples of vectors include plasmids, cosmids, bacmids, and viral vectors. Categories of viral vectors include bacteriophage (e.g., lambda phage, M13 phage), retrovirus (including lentivirus), adenovirus, adeno-associated virus, herpesvirus (e.g., herpes simplex virus), poxvirus, baculovirus, papillomavirus, and papovavirus (e.g., SV40). In a preferred embodiment, the vector is a lentivirus such as human immunodeficiency virus (HIV). A vector may contain a variety of elements for controlling expression of the polynucleotide sequence of interest, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The terms “introduced,” “introduced,” “introducing,” and “introduction” as used herein refer to the entry of a vector or vectors into a host cell. A vector may be introduced into the host cell by non-viral transfection methods or by viral transduction. Non-viral transfection methods include but are not limited to DEAE-Dextran-mediated, calcium-phosphate mediated, cationic lipid-mediated transfection, electroporation, nucleofection, lipofection, microinjection, ballistic introduction, and scrape loading. In a preferred embodiment, a vector is introduced into a host cell by viral transduction, preferably using a lentiviral vector.

The term “variant” as used herein refers to a polypeptide that differs from a reference polypeptide but retains essential properties. For example, a Cre variant polypeptide may differ from Cre with regards to specific amino acid sequence, but will maintain the recombinase activity of Cre. Generally, differences are limited so that the sequences of the reference polypeptide and the variant are closely similar overall and, in many regions, identical. A variant and reference polypeptide may differ in amino acid sequence by one or more substitutions, additions, or deletions in any combination. A substituted or inserted amino acid residue may or may not be one encoded by the genetic code. A variant of a polypeptide may be naturally occurring or it may be a variant that is not known to occur naturally.

The phrase “renal toxicity” as used herein refers to a detrimental effect on the viability of renal cells, particularly renal proximal tubule epithelial cells. This detrimental effect may be initiation of cell senescence, or it may be a lesser effect such as initiating a decrease in normal cellular function or activity, for instance by increasing or decreasing protein expression or secretion.

The phrase “artificial renal tubule device” refers to a device designed to mimic the function of the renal tubules. Such a device may be incorporated into an artificial kidney dialysis device, where it is linked to a hemofilter for filtering excess solutes and water from the blood of a subject. An artificial kidney dialysis device may be external, in which case blood is removed from a subject via a means such as a catheter prior to passing through the device, or it may be internally implanted into the subject.

### Description

Primary human RPTECs are of limited use in basic research and clinical applications due to their limited life span. These cells have limited proliferation potential in culture, and undergo replicative senescence with extended passages. Previous attempts have been made to establish immortalized renal tubule cells using either a hybrid adeno 12-SV40 virus or the E6/E7 genes of HPV 16 (Ryan 1994; Racusen 1997). However, none of the clones derived from these methods formed “domes” in culture dishes, suggesting either a defect in directional flux of transepithelial solutes and fluid or the absence of intact cell-to-cell tight junctions in these cells.

In certain aspects, the present invention is drawn to a method for reversibly immortalizing human RPTECs and to the cells generated by this method. RPTECs are immortalized by introduction of a vector or vectors containing polynucleotide sequences encoding hTERT and Tag. The polynucleotide sequences encoding hTERT and Tag are flanked by loxP sites, allowing for removal of hTERT and Tag in the presence of Cre or a Cre variant. Cre, a 38-kDa recombinase from bacteriophage P1, utilizes its endonuclease activity to catalyze recombination between two identical loxP sites. The enzyme requires no accessory proteins or cofactors and functions efficiently in vitro and under a wide variety of cellular conditions (Abremski 1983; Sternberg 1981a; Sternberg 1981b; Sternberg 1981c). The recombination site recognized by Cre is a 34-base pair (bp) double-stranded DNA sequence known as loxP. Each loxP site consists of two 13-bp inverted repeats separated by an 8-bp asymmetrical core region. Cre binds to the inverted repeats and cleaves the DNA in the core region to facilitate DNA strand exchange reactions (Abremski 1983; Sternberg 1981a; Sternberg 1981b; Sternberg 1981c).

The polynucleotides encoding hTERT and Tag may both be contained in a single vector, or they may be contained in separate vectors. If they are contained in separate vectors, the vectors may be introduced into RPTECs simultaneously or sequentially. Sequential introduction of the vectors may be done in either order (i.e., hTERT followed by Tag or Tag followed by hTERT).

Once immortalized, stable clones generated by this method can be passaged for at least two months in culture. In a preferred embodiment these stable clones can be passaged in culture for at least six months, preferably at least one year, and most preferably at least two years. These stable clones continue to possess high levels of the functions and characteristics associated with RPTECs. The immortalization process can be reversed by introducing a vector containing a polynucleotide encoding Cre or a Cre variant into the cells. When the hTERT and Tag cDNAs are removed from the cells by Cre-mediated recombination, the renal cell-specific activities of the clones are generally upregulated. Removal of the genes responsible for cell immortalization also significantly decreases the rate of cell proliferation, and ultimately leads to cell senescence. This suggests that cell cycle control remains intact despite numerous passages of the cells in culture.

The reversibly immortalized RPTECs of the present invention differ significantly from RPTEC clones described previously (Ryan 1994; Racusen 1997). Unlike clones established by transduction of RPTECs with adeno 12-SV40 or HPV E6/E7, the clones described herein display reasonably high levels of brush border enzyme activity and Na⁺-dependent glucose uptake. Cell morphology, biochemical activity, and functional activity of the RPTECs of the present invention remains relatively unchanged with extended passage in cell culture. Telomerase expression has been proposed to increase genetic stability in a manner that is independent of regulating telomere length (Sharma 2003).

Removal of the SV40 Tag and hTERT cDNAs from the cells of the present invention by Cre-mediated recombination results in upregulation of renal cell-specific activities. This is in agreement with the general observation that proliferating cells express lower levels of tissue-specific functions. Removal of the genes responsible for cell immortalization also significantly decreases the rate of cell proliferation and ultimately leads to cell senescence, suggesting that cell cycle control remains intact despite continuous passage of these cells in culture.

The methods provided herein represent a general approach for establishing stable RPTEC lines that retain high-level tissue specific function while proliferating continuously in culture. As known in the art, the use of primary cells in applications such as drug screening, tissue replacement, tissue regeneration, and basic research is limited by a shortage of primary cells. The reversibly immortalized RPTECs of the present invention may be substituted for primary cells in a variety of applications. The ability of these cells to proliferate for over two years in culture allows prolonged expansion and ample supply of these cells.

In one embodiment, the reversibly immortalized RPTECs of the present invention may be used to screen a test agent to determine whether the test agent modulates renal toxicity. The cells of the invention may be contacted with the test agent, and then monitored for changes in proliferation, viability, or function. Many drugs and other agents induce toxicity in kidney cells. The cells of the present invention are ideal surrogates for in vitro testing of renal toxicity prior to animal testing. The toxicity of a test agent can be determined by contacting the agent with the RPTECs of the present invention and monitoring the RPTECs for changes in proliferation, viability, or function. A decrease in any of these characteristics may be indicative of renal toxicity. Test agents may also be tested for their ability to slow down or reverse renal toxicity induced by other agents. For example, an agent known to induce renal toxicity can be contacted to RPTECs either before or after introduction of vectors encoding hTERT and Tag. The immortalized cells of the invention may then be contacted with the test agent, and monitored for changes in proliferation, viability, or function. Increases in any of these characteristics may indicate that the test agent reduces or reverses renal toxicity. After agents are identified that modulate renal toxicity, the cells of the present invention may be used to study the mechanisms of renal toxicity.

Proliferation refers to an increase in the number of cells as a result of cell growth and division. Possible methods for monitoring proliferation include, but are not limited to, detection of an antigen associated with proliferation, measurement of DNA synthesis, and detection of reduction of the intracellular environment. DNA synthesis may be measured by, for instance, quantitating ³H-thymidine incorporation or 5-bromodeoxyuridine (BrdU) incorporation. Reduction of the intracellular environment may be monitored by tetrazolium salt reduction.

Viability refers to the ability of a cell or cells to survive and reproduce. Commonly used assays for measuring cell viability include, but are not limited to, Trypan Blue exclusion, Neutral Red staining, crystal violet inclusion, and ⁵¹Cr release. Test agents that induce renal toxicity may induce senescence in all immortalized RPTECs, or they may merely decrease viability of some subset of those cells.

Changes in the function of immortalized RPTECs may be manifested in a variety of ways. For example, a change in function may be indicated by a change in metabolic activity, such as an increase or decrease in glucose transport or ammonia production. A change in function may also be indicated by a change in the expression pattern of the immortalized RPTECs, such as the increase or decrease of expression of specific genes associated with renal toxicity. In addition, a change in function may be indicated by a change in the secretion pattern of the immortalized RPTECs, such as the increase or decrease of secretion of various proteins, signaling compounds, or other molecules. Each of these manifestations of altered function may be readily monitored or measured by one skilled in the art.

The observation that Cre recombinase treatment leads to the cell senescence phenotype in the cells of the present invention suggests that the cells may be used to screen test agents or genes associated with the upregulation or downregulation of cell senescence. For example, the immortalized RPTECs may be contacted with a test agent prior to Cre treatment, and it may be determined whether the test agent leads to cell senescence or tumorigenic conversion. Likewise, the immortalized RPTECs may be contacted with a test agent after Cre treatment, and it may be determined whether the test agent regulates (either by enhancing or suppressing) the cell senescence process.

The immortalized RPTECs of the present invention may be incorporated into an artificial kidney dialysis device or artificial renal tubule device for use in a subject with chronic renal failure or acute tubular necrosis. Basic dialysis devices are capable of filtering excess solutes and water from the blood, but lack the ability to replace kidney function entirely because they are unable to secrete the endocrine and immunologic factors normally secreted by the kidney. This has led to the development of artificial kidney dialysis devices and artificial renal tubule devices capable of replacing the metabolic and cellular functions that reside in the tubular compartments of the kidney (Humes 1999). These devices incorporate primary RPTECs into hollow fibers that mimic the function of the renal proximal tubules (Humes 1999; Fissell 2003). One drawback of using primary RPTECs in these devices is that the cells will expand and their functional characteristics will change over time. These problems can be overcome in part by using the reversibly immortalized RPTECs of the present invention rather than primary RPTECs. Thus, the present invention contemplates the use of reversibly immortalized RPTECs in a device designed to mimic the function of the renal proximal tubules.

The immortalized RPTECs of the present invention may be transplanted into a subject exhibiting decreased or faulty renal function. The immortalized RPTECs may be transplanted alone, or in conjunction with various non-immortalized cells. The use of immortalized cells provides a means to overcome the shortage of primary RPTECs available for such procedures.

Immortalized RPTECs in which immortalization has been reversed by introduction of Cre exhibit decreased growth and proliferation. After 2-3 weeks, the Cre-treated cells develop morphology consistent with cell senescence, suggesting that regulation of the cell cycle remains intact in the immortalized cells. In addition, RPTECs in which immortalization has been reversed exhibit increased differentiation functions and glucose transport. As with the immortalized RPTECs described above, RPTECs in which immortalization has been reversed may be used to screen test agents for the ability to modulate renal toxicity. In addition, these cells may be incorporated into an artificial renal tubule or kidney dialysis device, or transplanted into a subject exhibiting decreased or faulty renal function.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Generation of HIV-Based Vectors Containing hTERT or SV40 Tag

Two HIV-based vectors containing the neo^{R} gene together with either SV40 Tag or hTERT cDNA were generated. Both vectors contained a gene cassette flanked by two loxP sites and placed under the control of the CMV IE promoter.

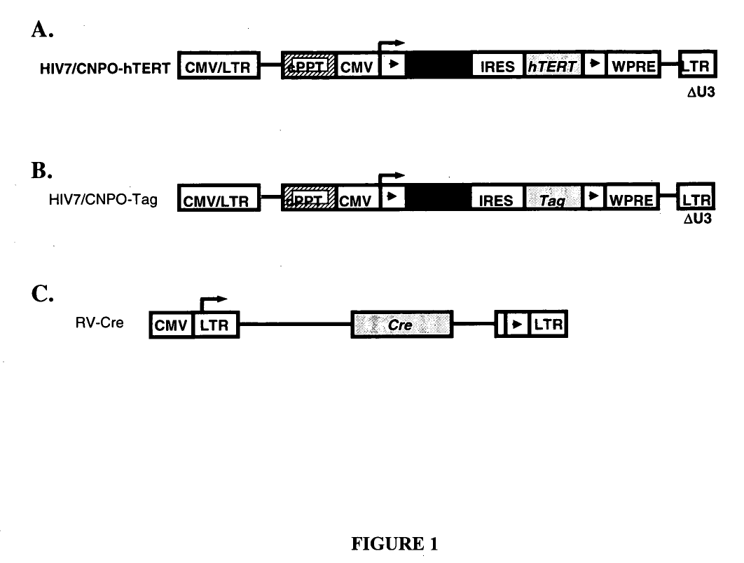

pHIV7/CNPO was constructed by inserting a 2,400-bp Notl/HindIII fragment into the unique BamHl site of pHIV7 (Kowolik 2000). The 2,400-bp insert contained the neomycin resistance (neo^{R}) gene and an internal ribosome entry site (IRES) flanked by two loxP sites controlled by the immediate early (IE) gene promoter of cytomegalovirus (CMV). pHIV7/CNPO-hTERT (FIG. 1A) was constructed by inserting a 3,490-bp EcoRI/Sall fragment containing the cDNA sequence for hTERT into the BamHl site of pHIV7/CNPO. pHIV7/CNPO-Tag (FIG. 1B) was constructed by inserting a 2,170-bp BamHl fragment containing the cDNA sequence for SV40 Tag into the BamHI site of pHIV7/CNPO.

HIV vectors were produced from 293T cells by transient transfection and the titer was determined in HT1080 cells as described previously (Kowolik 2002). Both cell types were maintained in Dulbecco's Modified Eagle's Medium (DMEM) supplemented with 10% fetal calf serum (FCS).

### Example 2

### Generation of Immortalized RPTEC Clones Expressing hTERT and SV40 Tag

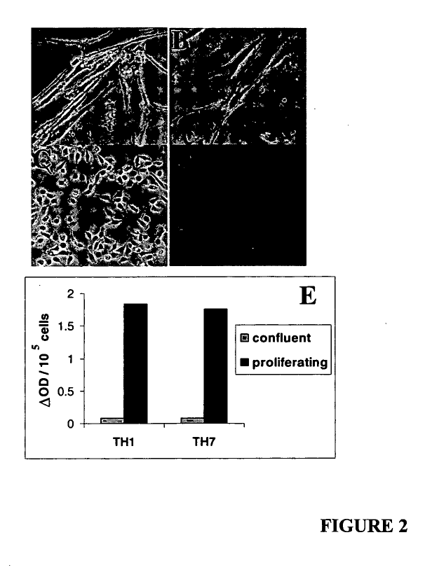

Primary human renal proximal tubule epithelial cells (RPTEC) were purchased from Clonetics (Walkersville, Md.) and maintained in Renal Epithelial Cell Growth medium (REGM). These primary human RPTECs underwent replicative senescence at passage seven. At this point, RPTECs stopped dividing and became elongated (FIG. 2A). Expression of the senescence specific biomarker β-gal was detectable in many of the elongated cells (FIG. 2B).

To establish immortalized clones, RPTECs from the third and fourth passages were transduced with both HIV-7/CNPO-Tag and HIV-7/CNPO-hTERT vectors at a multiplicity of infection (MOI) of one, followed by selection with G418. Neo^{R} clones were picked and expanded. The immortalized cells continued to proliferate for more than 100 passages (>2 years) in culture, and the doubling time for these cells lines was in the range of 40 to 48 hours. Cells from one of these clones at passage 30 are shown in FIG. 2C. When treated with an antibody specific for cytokeratin, an intermediate filament found in epithelial cells, all cells stained positively (FIG. 2D). The same antibody failed to stain HT1080 cells, a human fibrosarcoma line.

To examine cell proliferation, cells were either plated at a density of 5×10⁴ cells/well in a 24-well plate or kept in confluent cultures for three days. Cell proliferation was determined using the BrdU cell proliferation kit from Oncogene (San Diego, Calif.), and results were normalized to the cell number. Cell growth required the continuous presence of growth factors in 0.5% FCS-containing media and was contact inhibited as determined by BrdU incorporation (FIG. 2E). BrdU labeling in proliferating cells was 20-fold more efficient than in confluent cells, suggesting that cell growth was contact-inhibited. The immortalized cells failed to form anchorage-independent colonies in soft agar. They also failed to form tumors in nude mice after subcutaneous injection of 5×10⁶ cells/mouse. These results strongly suggest that the immortalized cells were not transformed.

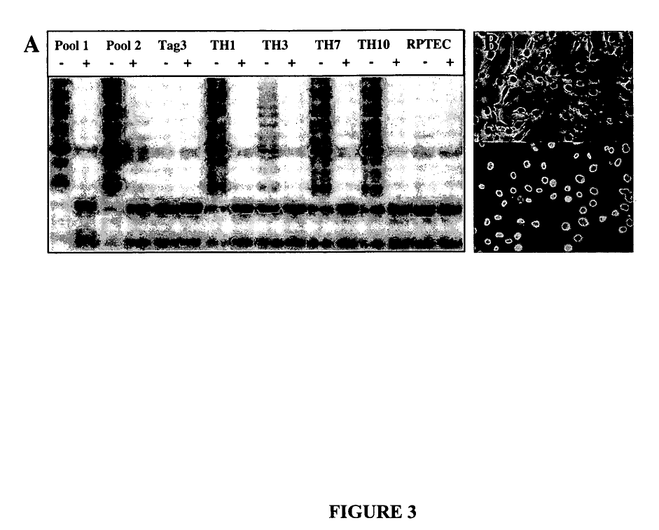

To confirm hTERT expression, cell extracts from both immortalized clones and primary RPTECs were subjected to a telomerase activity assay using the Trapeze telomerase detection kit (Intergen, Purchase, N.Y.). Telomerase activity was detected in two different pools of approximately 500 Neo^{R} colonies derived from co-transduction with both vectors (FIG. 3A, “pool 1” and “pool 2”). Individual clones isolated from the same transduction were also positive for telomerase activity (FIG. 3A, “TH1,” “TH3,” “TH7,” TH10”). In each of these samples, telomerase activity disappeared when the cell extract was subjected to heat treatment at 85° C. for ten minutes (FIG. 3A). In contrast, a stable clone derived from transduction with the Tag vector only displayed no detectable telomerase activity (FIG. 3A, “Tag3”). Thus, while Tag expression is sufficient to establish stable clones, it is not capable of activating endogenous telomerase expression. Parental RPTECs, like most primary cells, failed to express telomerase activity (FIG. 3A, “RPTEC”).

To confirm Tag expression, immortalized clones were treated with an anti-Tag antibody followed by immunofluorescent staining. Cells were seeded in six-well plates at a density of 10⁵ cells/well, fixed with 2% formaldehyde at 4° C. for 30 minutes, and permeabilized with 0.2% Triton X-100 for 15 minutes at room temperature. Following three wash steps with PBS, non-specific binding sites were blocked for 30 minutes with 5% bovine serum albumin in PBS. The samples were subsequently incubated for two hours at room temperature with a 1:200 dilution of a mouse anti-Tag antibody (Santa Cruz Biotechnology, Santa Cruz, Calif.) in blocking solution. After washing the samples three times with PBS, they were incubated for one hour with 1 μg/ml FITC-labeled goat anti-mouse antibody (Sigma, St. Louis, Mo.) in blocking solution. Following three additional wash steps, the samples were visualized by fluorescence microscopy.

TH1 cells expressed Tag in the nuclei (FIG. 3B). A control antibody gave only background staining (data not shown). These results suggest that co-expression of hTERT and Tag efficiently immortalizes primary human RPTECs and generates stable clones that can be passaged in culture for more than 2 years.

### Example 3

### Morphological Characteristics of Immortalized RPTECs

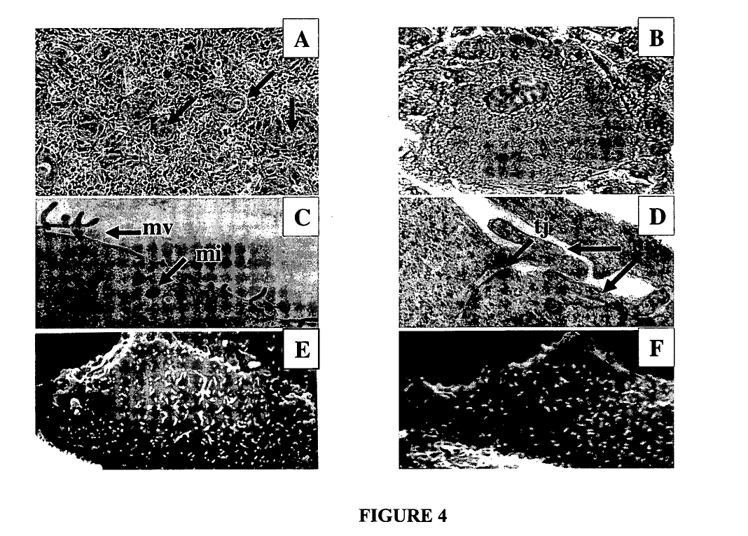

Individual clones TH1 and TH7 were selected for further study. They grew as a monolayer with a cobblestone appearance (FIG. 4A). Multiple “dome” formation, which is one of the characteristics of primary RPTECs grown in culture (Dreher 1992), was consistently observed in a confluent culture (FIG. 4A, arrows, and 4B). Formation of these structures is likely due to transepithelial transport of water and solutes trapped between the cultured cell layer and the culture dish. The presence of domes in confluent TH1 and TH7 cultures suggests that the transportation function for water and solutes remains intact in the immortalized cell lines. Ultrastructural examination of the immortalized cells by electron microscopy indicated the presence of short and long microvilli at their apical surface, facing the culture medium (FIGS. 4C-4F). The density of microvilli remained relatively unaltered irrespective over extended passages (FIGS. 4E-4F), suggesting that these clones were stable in culture. Tight junctions were numerous and preferentially located in the apical domain of the lateral border (FIG. 4D). Numerous mitochondria and ribosomes were randomly scattered throughout cytoplasm (FIG. 4C). This confirms that the immortalized cells exhibited the characteristic morphology of RPTECs.

### Example 4

### Generation of a Retroviral Vector Containing Cre

A retroviral vector, RV-cre, containing the cre gene and the LoxP site in both LTRs was generated as described previously (Silver 2001). To construct the pRV-cre plasmid, a 1,100-bp HindIII/EcoRI fragment containing the cre gene was inserted into the unique C/al of pCMV-LL-SA-2-lox. pCMV-LL-SA-2-lox is a murine leukemia virus (MLV)-based retroviral vector containing a loxP site in the U3 region of the 3'LTR.

Since RV-cre contained no selectable marker, a stable cell line, PCCG-β-gal, was established to determine the titer of RV-cre. HT1080 cells were transduced with an MLV-based retroviral vector containing the GFP gene flanked by two loxP sites, followed by the E. coli β-galactosidase (lacZ) gene. The expression of both GFP and lacZ was under the control of the CMV IE promoter. However β-gal production was absent due to the translational block by the termination codon of the upstream GFP gene. Deletion of the GFP gene by Cre removed this block and allowed β-gal expression. The transduced cells were sorted for GFP expression and a single clone, PCCG-β-gal, was obtained by limiting dilution. To determine the RV-cre titer, 10⁵ PCCG-β-gal cells were transduced and stained for β-gal expression 72 hours after transduction.

### Example 5

### Removal of SV40 Tag and hTERT cDNAs by Cre-Mediated Recombination

Both the SV40 Tag and hTERT cDNAs are flanked by LoxP sites and can be removed by the Cre recombinase. Since stable Cre expression induces toxicity in mammalian cells (Silver 2001; Pfeifer 2001), a transient Cre expression system was used to remove the Tag and hTERT cDNAs from the immortalized TH clones.

Expression of the cre gene in transduced cells is expected to remove both the target gene and the cre gene itself. To remove the Tag and hTERT cDNAs, immortalized cells were transduced three times with RV-cre at a MOI of two, followed by staining with a Tag specific antibody ten days later.

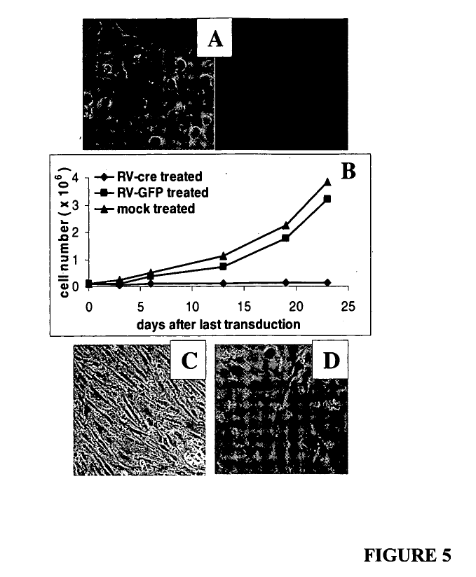

No nuclear staining of Tag could be detected in RV-cre transduced TH₁ cells (FIG. 5A). To determine the effect of Cre treatment on the immortalized cells, cell proliferation of mock-transduced, RV-cre, and RV-GFP (a GFP-containing retroviral vector) transduced cells in culture was measured. Cell proliferation was determined using the BrdU cell proliferation kit. The growth rate of the RV-cre transduced cells was significantly reduced when compared with that of the mock or RV-GFP transduced cells (FIG. 5B). RV-cre transduced cells continued to proliferate, albeit at an extremely slow rate. At 2-3 weeks after the initial transduction, RV-cre cells ceased proliferation and changed cell morphology (FIG. 5C). These changes were reminiscent of cell senescence of primary RPTECs (FIG. 2A). Elongated cells were stained for senescence-specific β-gal. The cells were fixed with 3% formaldehyde, washed with PBS, and incubated at 37° C. with freshly prepared staining solution (1 mg/ml 5-bromo-4-chloro-3-indolyl β-D-galactoside in 40 mM citric acid, sodium phosphate, pH 6.0, 5 mM potassium ferrocyanide, 5 mM potassium ferricyanide, 150 mM NaCl, 2 mM MgCl₂) (Dimri 1995). The reaction was stopped after four hours. Many of the RV-cre transduced cells expressed β-gal (FIG. 5D). Identical results were obtained with TH7 cells. This suggests that despite continuous passage of TH1 and TH7 cells in culture for more than two years, the cell cycle control remains intact in these cells.

### Example 6

### Biochemical Properties of Immortalized RPTECs

The activity of two brush-border enzymes, alkaline phosphatase (AP) and y-glutamyl transpeptidase (GGT), was measured in TH1 and TH7 cells. AP and GGT serve as markers for proximal tubule differentiation. These two enzymes were assayed because their activities were affected most severely in human RPTEC clones established previously by the expression of Tag alone (Racusen 1997).

Cell extracts were assayed for GGT activity by measuring p-nitroaniline liberation from the substrate y-glutamyl-p-nitroanilide (Sigma, St. Louis, Mo.) at 405 nm (De Young 1978). Cell extracts were assayed for AP activity was determined by measuring p-nitrophenol liberation from the substrate p-nitrophenylphosphate (Sigma, St. Louis, Mo.) at 405 nm (Rudolph 1999). The total protein concentration of the cell extract was determined using the Coomassie Protein Reagent Assay Reagent (Pierce, Rockford, Ill.).

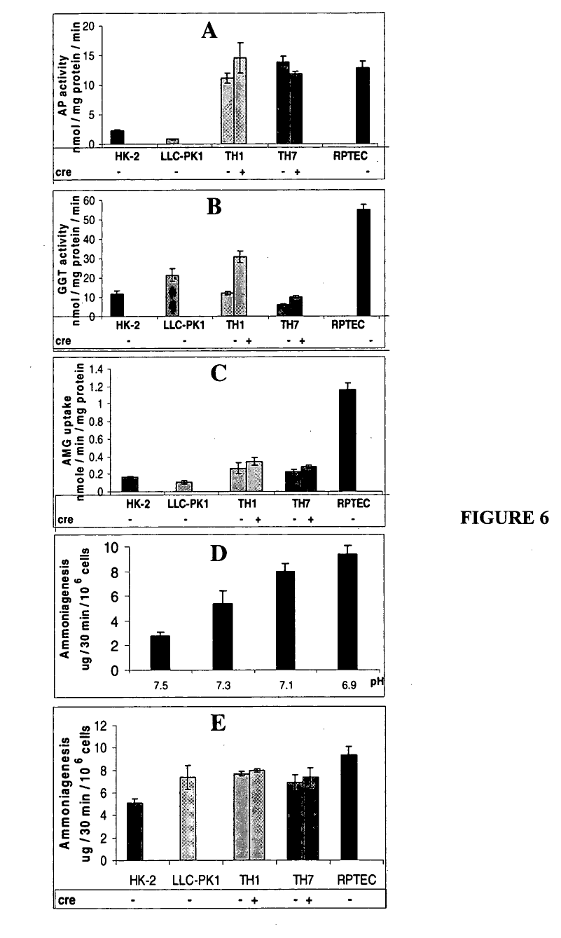

AP activity in TH1 and TH7 cells ranged from 10 to 15 nmole/min/mg protein (FIG. 6A). These results were similar to those seen in cultured primary human RPTECs. AP activity in the TH1 and TH7 clones was at least 4-5 fold higher than that previously reported for immortalized clones established using SV40 Tag alone (Racusen 1997). GGT activity in TH1 and TH7 cells was 12 and 6 nmol/min/mg protein, respectively (FIG. 6B). While this is lower than the activity reported for primary RPTECS, it is at least 4-8 fold higher than the activity for clones immortalized with SV40 Tag alone (Racusen 1997).

To determine the effect of Cre-mediated gene removal on AP and GGT activity, TH1 and TH7 cells were analyzed ten days after RV-cre treatment. AP activity increased slightly in TH1, but decreased slightly in TH7 (FIG. 6A). The differences were statistically insignificant. This was expected since the levels of AP in these two clones were already near the level in primary cells prior to RV-cre treatment. In contrast, GGT activity increased three-fold in TH1 and more than 60% in TH7 following RV-cre treatment (FIG. 6B). This suggests that some differentiation functions were upregulated significantly after removal of the genes responsible for cell immortalization. Extended passage of TH1 and TH7 cells in culture did not cause significant changes in either AP or GGT activity. The relatively stable activities of the brush border enzymes in these two clones were consistent with stable microvilli density irrespective of the passage number in culture (FIG. 4E-4F).

Together, these results suggest that the participation of hTERT in the cell immortalization process may facilitate the establishment of clones that are phenotypically more stable and express significantly higher levels of renal tubule cell differentiation functions than immortalized clones established by SV40 Tag alone.

### Example 7

### Functional Properties of Immortalized RPTECs

Renal proximal tubule cells perform critical metabolic activities, including active glucose transport and ammonia production and excretion. To characterize glucose transport in immortalized cells, α-methylglucopyranoside (AMG) uptake was measured in the presence and absence of phlorizin, a potent inhibitor of the Na⁺-dependent glucose co-transporter (Rabito 1980). The uptake assay was performed with α-[¹⁴ C]-AMG (NEN, Boston, Mass.) as described previously (Joly 1990). Radioactive counts were normalized to protein content as determined using the Coomassie Protein Assay Reagent (Pierce, Rockford, Ill.). The specific AMG uptake results from the difference between AMG uptake in the presence and absence of phlorizin. As a comparison, glucose uptake was also measured in two well-established renal tubule cell lines, porcine LLC-PK1 and human HK-2 immortalized using the E6/E7 genes of the human papilloma virus (Ryan 1994). HK-2 and LLC-PK1 cells were purchased from American Type Culture Collection (Manassas, Va.). HK-2 cells were maintained in REGM, while LLC-PK1 cells were maintained in Medium 199 (Invitrogen, Carlsbad, Calif.) supplemented with 1.5 g/l sodium bicarbonate and 3% FCS.

Na⁺-dependent AMG uptake in TH1 and TH7 cells was 0.27 and 0.23 nmole/min/mg protein, respectively, about four-fold lower than in primary cells (FIG. 6C). AMG uptake was 30-60% higher in TH cells than in HK-2 cells, and about 1.5-fold higher in TH cells than in LLC-PK1 cells (FIG. 6C). To determine the effect of gene removal, AMG uptake was measured ten days after RV-cre treatment. AMG uptake increased approximately 30% in TH cells (FIG. 6C). This increase is statistically significant (p<0.05, T-test). Thus, removal of the genes used for cell immortalization upregulates active glucose transport function in the immortalized cells.

Proximal tubule cells respond rapidly to acute declines in blood pH by enhancing ammonia production and excretion. To measure this function in TH cells, ammonia production was measured at different pH levels. Culture medium was adjusted to various pH levels using HCI, and cells were incubated with fresh medium ranging from pH 7.5 to 6.9. Samples were collected after 30 minutes, and ammonia concentration was determined using an ammonia assay kit (DCL, Oxford, Conn.). Results were normalized to the number of cells per sample.

Primary RPTECs demonstrated gradual increases in ammonia production in response to media pH changes from pH 7.5 to 6.9 (FIG. 6D). Similar results were obtained with the other cell lines used in this study. A maximum level of approximately 7.7 and 6.9 μg/30 min/10⁶ cells was observed in TH1 and TH7 at pH 6.9, respectively (FIG. 6E). Primary RPTECs produced a slightly higher level at 9 μg/30 min/10⁶ cells. LLC-PK1 cells produced ammonia at levels comparable to TH cells, while HK2 cells produced only 65% the level seen in TH1 cells. No significant change in ammonia production could be detected after RV-cre treatment of TH1 and TH7 cells (FIG. 6E).

As stated above, the foregoing are merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### References

1. Abremski, K., Hoess, R., Sternberg, N. 1983. Studies on the properties of P1 site-specific recombination: evidence for topologically unlinked products following recombination. Cell 32:1301-1311.
2. Berghella, L., et al. 1999. Reversible immortalization of human myogenic cells by site-specific excision of a retrovirally trasferred oncogene. Hum Gene Ther 10:1607-1617.
3. Bodnar, A. G., et al. 1998. Extension of life-span by introduction of telomerase into normal human cells. Science 279:349-352.
4. Burger, A. M., et al. 1998. Effect of oncogene expression on telomerase activation and telomere length in human endothelial, fibroblast and prostate epithelial cells. Int J Oncol 13:1043-1048.
5. Condon, J., et al. 2002. Telomerase immortalization of human myometrial cells. Biol Reprod 67:506-514.
6. De Young, L. M., et al. 1978. Localization and significance of gamma-glutamyltranspeptidase in normal and neoplastic mouse skin. Cancer Res 38:3697-3701.
7. Dimri, G. P., et al. 1995. A biomarker that identifies senescent human cells in culture and in aging skin in vivo. Proc Natl Acad Sci USA 92:9363-9367.
8. DiRenzo, J., et al. 2002. Growth factor requirements and basal phenotype of an immortalized mammary epithelial cell line. Cancer Res 62:89-98.
9. Dreher, D., Rochat, T. 1992. Hyperoxia induces alkalinization and dome formation in MDCK epithelial cells. Am J Physiol 262: C358-364.
10. Fissell, W. H., et al. 2003. Bioartificial kidney ameliorates gram-negative bacteria-induced septic shock in uremic animals. J Am Soc Nephrol 14:454-461.
11. Hahn, W. C., et al. 1999. Creation of human tumour cells with defined genetic elements. Nature 400:464-468.
12. Harley, C. B., Futcher, A. B., Greider, C. W. 1990. Telomeres shorten during aging of human fibroblasts. Nature 345:458-460.
13. Herbert, B. S., Wright, W. E., Shay, J. W. 2002. p16(INK4a) inactivation is not required to immortalize human mammary epithelial cells. Oncogene 21:7897-7900.
14. Humes, H. D., et al. 1999. Replacement of renal function in uremic animals with a tissue-engineered kidney. Nature Biotech 17:451-455.
15. Hurlin, P. J., Maher, V. M., McCormick, J. J. 1989. Malignant transformation of human fibroblasts caused by expression of a transfected T24 HRAS gene. Proc Natl Acad Sci USA 86:187-191.
16. Jiang, X. R., et al. 1999. Telomerase expression in human somatic cells does not induce changes associated with a transformed phenotype. Nat Genet 21:111-114.
17. Joly, V., Saint-Julien, L., Carbon, C., Yeni, P.1990. Interactions of free and liposomal amphotericin B with renal proximal tubular cells in primary culture. J Pharmacol Exp Ther 255:17-22.
18. Kawano, Y., et al. 2003. Ex vivo expansion of human umbilical cord hematopoietic progenitor cells using a coculture system with human telomerase catalytic subunit (hTERT)-transfected human stromal cells. Blood 101:532-540.
19. Kim, B. H., et al. 2000. Dedifferentiation of conditionally immortalized hepatocytes with long-term in vitro passage. Exp Mol Med 32:29-37.
20. Kim, H., et al. 2002. Events in the immortalizing process of primary human mammary epithelial cells by the catalytic subunit of human telomerase. Biochem J 365:765-772.
21. Kiyono, T., et al. 1991. Both Rb/p 16INK4a inactivation and telomerase activity are required to immortalize human epithelial cells. Nature 396:84-88.
22. Kobayashi, N., et al. 2000a. Reversible immortalization of adult human hepatocytes. Transplant Proc 32:2331-2332.
23. Kobayashi, N., et al. 2000b. Prevention of acute liver failure in rats with reversibly immortalized human hepatocytes. Science 287:1258-1262.
24. Kowolik, C. M., Yee, J. K. 2002. Preferential transduction of human hepatocytes with lentiviral vectors pseudotypes by Sendai virus F protein. Mol Ther 5:762-769.
25. Land, H., Parada, L. F., Weinberg, R. A. 1983. Tumorigenic conversion of primary embryo fibroblasts requires at least two cooperating oncogenes. Nature 304:596-602.
26. Lundberg, A. S., et al. 2002. Immortalization and transformation of primary human airway epithelial cells by gene transfer. Oncogene 21:4577-4586.
27. Macieira-Coelho, A., Azzarone, B. 1988. The transition from primary culture to spontaneous immortalization in mouse fibroblast populations. Anticancer Res 8:669-676.
28. Meyerson, M., et al. 1997. hEST2, the putative human telomerase catalytic subunit gene, is up-regulated in tumor cells and during immortalization. Cell 90:785-795.
29. Morales, C. P., et al. 1999. Absence of cancer-associated changes in human fibroblasts immortalized with telomerase. Nat Genet 21:115-118.
30. Noguchi, H., et al. 2002. Controlled expansion of human endothelial cell populations by Cre-loxP-based reversible immortalization. Hum Gene Ther 13:321-334.
31. O'Hare, M. J., et al. 2001. Conditional immortalization of freshly isolated human mammary fibroblasts and endothelial cells. Proc Natl Acad Sci USA 98:646-651.
32. Pfeifer, A., et al. 2001. Delivery of the Cre recombinase by a self-deleting lentiviral vector: efficient gene targeting in vivo. Proc Natl Acad Sci USA 98:11450-11455.
33. Prowse, K. R., Greider, C. W. 1995. Developmental and tissue-specific regulation of mouse telomerase and telomere length. Proc Natl Acad Sci USA 92:4818-4822.
34. Rabito, C. A., Ausiello, D. A. 1980. Na+-dependent sugar transport in a cultured epithelial cell line from pig kidney. J Membr Biol 54:31-38.
35. Racusen, L. C. 1995. The histopathology of acute renal failure. New Horiz 3:662-668.
36. Racusen, L. C., et al. 1997. Cell lines with extended in vitro growth potential from human renal proximal tubule: characterization, response to inducers, and comparison with established cell lines. J Lab Clin Med 129:318-329.
37. Rhim, J. S., et al. 1985. Neoplastic transformation of human epidermal keratinocytes by AD12-SV40 and Kirsten sarcoma viruses. Science 227:1250-1252.
38. Rudolph, A. E., et al. 1999. Expression, characterization, and mutagenesis of the Yersinia pestis murine toxin, a phospholipase D superfamily member. J Biol Chem 274:11824-11831.
39. Ruley, H. E. 1983. Adenovirus early region 1A enables viral and cellular transforming genes to transform primary cells in culture. Nature 304:602-606.
40. Ryan, M. J., et al. 1994. HK-2: an immortalized proximal tubule epithelial cell line from normal adult human kidney. Kidney Int 45:48-57.
41. Sachsinger, J., et al. 2001. Telomerase inhibition in RenCa, a murine tumor cell line with short telomeres, by overexpression of a dominant negative mTERT mutant, reveals fundamental differences in telomerase regulation between human and murine cells. Cancer Res 61:5580-5586.
42. Salmon, P., et al. 2000. Reversible immortalization of human primary cells by lentivector-mediated transfer of specific genes. Mol Ther 2:404-414.
43. Sharma, G. G., et al. 2003. hTERT associates with human telomeres and enhances genomic stability and DNA repair. Oncogene 22:131-146.
44. Shay, J. W., Van Der Haegen, B. A., Ying, Y., Wright, W. E. 1993. The frequency of immortalization of human fibroblasts and mammary epithelial cells transfected with SV40 large T-antigen. Exp Cell Res 209:45-52.
45. Silver, D. P., Livingston, D. M. 2001. Self-excising retroviral vectors encoding the Cre recombinase overcome Cre-mediated cellular toxicity. Mol Cell 8:233-243.
46. Sternberg, N. 1981a. Bacteriophage P1 site-specific recombination. III. Strand exchange during recombination at lox sites. J Mol Biol 150:603-608.
47. Sternberg, N., Hamilton, D. 1981b. Bacteriophage P1 site-specific recombination. I. Recombination between loxP sites. J Mol Biol 150:467-486.
48. Sternberg, N., Hamilton, D., Hoess, R. 1981c. Bacteriophage P1 site-specific recombination. II. Recombination between loxP and the bacterial chromosome. J Mol Biol 150:487-507.
49. Stevenson, M., Volsky, D. J. 1986. Activated v-myc and v-ras oncogenes do not transform normal human lymphocytes. Mol Cell Biol 6:3410-3417.
50. van de Water, B., Houtepen, F., Huigsloot, M., Tijdens, I. B. 2001. Suppression of chemically induced apoptosis but not necrosis of renal proximal tubular epithelial (LLC-PK1) cells by focal adhesion kinase (FAK). J Biol Chem 276:36183-36193.
51. Westerman, K. A., Leboulch, P. 1996. Reversible immortalization of mammalian cells mediated by retroviral transfer and site-specific recombination. Proc Natl Acad Sci USA 93:8971-8976.
52. Wright, W. E., Pereira-Smith, O. M., Shay, J. W. 1989. Reversible cellular senescence: implications for immortalization of normal human diploid fibroblasts. Mol Cell Biol 9:3088-3092.
53. Wright, W. E., Shay, J. W. 1992a. Telomere positional effects and the regulation of cellular senescence. Trends Genet 8:193-197.
54. Wright, W. E., Shay, J. W. 1992b. The two-stage mechanism controlling cellular senescence and immortalization. Exp Gerontol 27:383-389.
55. Yoakum, G. H., et al. 1985. Transformation of human bronchial epithelial cells transfected by Harvey ras oncogene. Science 227:1174-1179.

1. A method of reversibly immortalizing a human renal proximal tubule epithelial cell (RPTEC) comprising introducing a first vector and a second vector into said RPTEC, wherein said first vector contains a first polynucleotide sequence encoding human telomerase catalytic subunit (hTERT) and said second vector contains a second polynucleotide sequence encoding SV40 T antigen (Tag), and wherein said first and second polynucleotide sequences are each flanked by loxP sites.

2. The method of claim 1, wherein said first and second vectors are contained in a single vector.

3. The method of claim 1, wherein said first and second vectors are introduced into said RPTEC simultaneously or sequentially.

4. The method of claim 1, wherein said first and second vectors are retroviral vectors.

5. The method of claim 4, wherein said first and second vector are lentiviral vectors.

6. The method of claim 1, further comprising the step of introducing into said RPTEC a third vector containing a third polynucleotide sequence encoding Cre or a Cre variant.

7. The method of claim 6, wherein said Cre or a Cre variant is transiently expressed in said RPTEC.

8. The method of claim 6, wherein said third vector is a retroviral vector.

9. The method of claim 8, wherein said third vector is a lentiviral vector.

10. A reversibly immortalized human cell comprising a human renal proximal tubule epithelial cell (RPTEC) into which a first vector and a second vector have been introduced, wherein said first vector contains a first polynucleotide sequence encoding human telomerase catalytic subunit (hTERT) and said second vector contains a second polynucleotide sequence encoding SV40 T antigen (Tag), and wherein said first and second polynucleotide sequences are each flanked by loxP sites.

11. The reversibly immortalized human cell of claim 10, wherein said first and second vectors are contained in a single vector.

12. The reversibly immortalized human cell of claim 10, wherein said first and second vectors are retroviral vectors.

13. The reversibly immortalized human cell of claim 12, wherein said first and second vectors are lentiviral vectors.

14. The reversibly immortalized human cell of claim 10, wherein a third vector containing a third polynucleotide sequence encoding Cre or a Cre variant has been introduced into said reversibly immortalized human cell.

15. The reversibly immortalized human cell of claim 14, wherein said Cre or a Cre variant is transiently expressed in said reversibly immortalized human cell.

16. The reversibly immortalized human cell of claim 14, wherein said third vector is a retroviral vector.

17. The reversibly immortalized human cell of claim 16, wherein said third vector is a lentiviral vector.

18. The reversibly immortalized human cell of claim 10, wherein said reversibly immortalized human cell is used to screen a test agent to determine whether said test agent modulates renal toxicity.

19. The reversibly immortalized human cell of claim 10, wherein said reversibly immortalized cell is incorporated into an artificial renal tubule device.

20. A method of screening a test agent to determine whether said test agent modulates renal toxicity, comprising
a) contacting said test agent to one or more reversibly immortalized human cells, wherein each reversibly immortalized human cell comprises a human renal proximal tubule epithelial cell (RPTEC) into which a first vector and a second vector have been introduced, wherein said first vector contains a first polynucleotide sequence encoding human telomerase catalytic subunit (hTERT) and said second vector contains a second polynucleotide sequence encoding SV40 T antigen (Tag), and wherein said first and second polynucleotide sequences are each flanked by loxP sites; and
b) monitoring said one or more reversibly immortalized human cells for a change in proliferation, viability, or function, wherein said change indicates that said test agent modulates renal toxicity.
